(19) United States
(12) Patent Application Publication
(10) Pub. No.: US 2012/0219950 A1
(43) Pub. Date: Aug. 30, 2012

(54) ASSAY SYSTEMS FOR DETECTION OF ANEUPLOIDY AND SEX DETERMINATION

(76) Inventors: Arnold Oliphant, San Jose, CA (US); Ken Song, San Jose, CA (US)

(21) Appl. No.: 13/405,839

(22) Filed: Feb. 27, 2012

Related U.S. Application Data
(60) Provisional application No. 61/447,563, filed on Feb. 28, 2011.

(51) Int. Cl. C12Q 1/68

(52) U.S. Cl. 435/6.11

(57) ABSTRACT

The present invention utilizes detection of selected nucleic acid regions from pseudoautosomal regions to identify sex chromosomal aneuploidy and to determine fetal sex. Traditional methods of detecting sex chromosomal aneuploidies and performing sex determination typically involves some analysis of the Y chromosome. The assay systems of the present invention utilizing copy number variant detection of pseudoautosomal regions allows quantification of the sex chromosomes in mixed samples using loci that display autosomal inheritance patterns.

### RELATED APPLICATIONS

The present application claims priority to U.S. Ser. No. 61/447,563, filed Feb. 28, 2011, which is incorporated by reference.

### FIELD OF THE INVENTION

This invention relates to detection of sex chromosome copy number for detection of aneuploidies and sex determination.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The pseudoautosomal regions, PAR1 and PAR2, are homologous sequences of nucleotides on the X and Y chromosomes. Mangs A H and Morris B J, Curr Genomics. 2007 April; 8(2): 129-136. The pseudoautosomal regions obtained this name because any loci located within them are inherited in the same fashion as autosomal loci.

Normal male mammals have two copies of these loci: one in the pseudoautosomal region of their Y chromosome, the other in the corresponding portion of their X chromosome. Normal females also possess two copies of pseudoautosomal loci, as each of their two X chromosomes contains a pseudoautosomal region. Synapsis of the X and Y chromosomes and recombination between the X and Y chromosomes is normally restricted to the pseudoautosomal regions, and pseudoautosomal loci thus exhibit an autosomal, rather than sex-linked, pattern of inheritance.

PAR1 comprises 2.6 Mb of the short-arm tips of both X and Y chromosomes in humans and other great apes and PAR2 is located at the tips of the long arms, spanning 320 kb. The function of these pseudoautosomal regions is that they allow the X and Y chromosomes to pair and properly segregate during meiosis in males. Ciccodicola A, D'Esposito M, Esposito T, et al. (2000), Hum. Mol. Genet. 9 (3): 395-401. To date, at least 29 genes have been found within PAR1 and PAR2. Blaschke R J and Rappold G (2006), Curr Opin Genet Dev 16 (3): 23-9. All pseudoautosomal loci escape X-inactivation and are therefore candidates for having dosage effects in sex chromosome aneuploidy conditions.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides methods and assay systems that utilize detection of selected nucleic acid regions from mammalian pseudoautosomal regions (PARs) to identify sex chromosomal aneuploidy and/or to determine fetal sex. Traditional methods of detecting sex chromosomal aneuploidies and performing sex determination typically involves analysis of Y-specific sequences. The assay systems of the invention identify copy number variants of pseudoautosomal regions, allowing quantification of the sex chromosomes in mixed samples using loci from the X and/or Y chromosome that display autosomal inheritance patterns.

In one aspect, the invention provides methods for analysis of selected sequences within the PARs to determine whether an abnormal number of sex chromosomes are present in a biological sample. In the case of a normal male or female, two sets of PARs will be present. Abnormal sex chromosome copy number variants such as trisomy, tetraploidy and the like can be identified by detection of abnormal copy number of selected sequences within the PARs of a biological sample, e.g., the PARs of an individual genome or the PARs of a mixed sample. The detection of a copy number variant of all or part of a sex chromosome is performed using a comparison to a reference genomic region or regions (e.g., an autosomal region on a chromosome) which are normal in copy number.

In a general aspect, the invention provides an assay system for detection of the presence or absence of a sex chromosome aneuploidy comprising the steps of providing a biological sample containing DNA, amplifying one or more selected nucleic acid regions from a pseudoautosomal region in the biological sample, amplifying one or more selected nucleic acid regions from an autosomal region in the biological sample, detecting the amplified nucleic acid regions, quantifying the relative frequency of the selected nucleic acid regions from the pseudoautosomal and autosomal regions, comparing the relative frequency of the selected nucleic acid regions from the pseudoautosomal and autosomal regions; and identifying the presence or absence of an aneuploidy of a sex chromosome based on the compared relative frequencies of the pseudoautosomal and autosomal regions.

In one specific aspect, the invention provides an assay system for detection of the presence or absence of a sex chromosome aneuploidy comprising the steps of providing a mixed sample comprising cell free DNA, amplifying two or more selected nucleic acid regions from a pseudoautosomal region in the mixed sample, amplifying two or more selected nucleic acid regions from an autosomal region in the mixed sample, detecting the amplified nucleic acid regions, quantifying the relative frequency of the selected nucleic acid regions from the pseudoautosomal and autosomal regions, comparing the relative frequency of the selected nucleic acid regions from the pseudoautosomal and autosomal regions; and identifying the presence or absence of an aneuploidy of a sex chromosome in a cell population based on the compared relative frequencies of the pseudoautosomal and autosomal regions.

In another specific aspect, the invention provides an assay system for detection of the presence or absence of a sex chromosome aneuploidy comprising the steps of providing a mixed sample comprising cell free DNA, sequencing cell-free DNA from the mixed sample, analyzing the relative frequency of the selected nucleic acid regions from the pseudoautosomal and autosomal regions, comparing the relative frequency of the selected nucleic acid regions from the pseudoautosomal and autosomal regions; and identifying the presence or absence of an aneuploidy of a sex chromosome in a cell population based on the compared relative frequencies of the pseudoautosomal and autosomal regions. In a more specific aspect, the sequencing is next generation sequencing. In other more specific aspects, the sequencing is massively parallel sequencing. Such techniques are described, e.g., in U.S. Pat. Nos. 7,888,017 and 8,008,018.

In a more specific aspect, the invention provides an assay system for detection of the presence or absence of a fetal sex chromosome aneuploidy in a maternal sample, comprising the steps of providing a maternal sample comprising maternal and fetal cell free DNA, amplifying two or more selected nucleic acid regions from a pseudoautosomal region in the maternal sample, amplifying two or more selected nucleic acid regions from an autosomal region in the maternal sample, detecting the amplified nucleic acid regions, quantifying the relative frequency of the selected nucleic acid regions from the pseudoautosomal and autosomal regions, comparing the relative frequency of the selected nucleic acid regions from the pseudoautosomal and autosomal regions, and identifying the presence or absence of a fetal aneuploidy based on the compared relative frequencies of the selected nucleic acid regions.

In certain aspects, the relative frequencies of the selected nucleic acid regions are individually quantified, and the relative frequencies of the individual nucleic acid regions are compared to determine the presence or absence of a sex chromosome aneuploidy. In other certain aspects, the relative frequencies of the chromosomes are determined using selected sequences from pseudoautosomal and autosomal regions, and the frequencies are expressed as a chromosomal ratio. The quantified relative frequencies of the nucleic acid regions are used to determine a chromosome frequency of one or both of the sex chromosomes, and the presence or absence of an aneuploidy is determined based on the compared chromosome frequencies. In more specific aspects, the quantified relative frequencies of the selected nucleic acid regions are normalized following detection and prior to quantification.

In some aspects, the selected nucleic acid regions are associated with one or more identifying indices. The frequency of the selected nucleic acid regions can be determined through identification of the associated one or more indices, and the relative frequencies of each nucleic acid region for the sex chromosome and the reference chromosome are summed and the sums compared to calculate a chromosomal ratio. In specific aspects, the chromosomal ratio is compared to the mean chromosomal ratio from a normal population and the threshold for identifying the presence or absence of an aneuploidy is at least three times the chromosomal variation in a normal population.

In a preferred aspect the nucleic acid regions of the assay system are assayed in a single vessel. In a more preferred aspect, the nucleic acid regions undergo a universal amplification. In another preferred aspect, the pseudoautosomal and autosomal nucleic acid regions are each counted an average of at least 500 times.

In a separate aspect, the invention provides assay systems and methods that utilize detection of selected sequences within the PARs for determining sex chromosome copy number variants in the fetus from a maternal biological sample, e.g., maternal blood, plasma or serum. In the case where the maternal biological sample is blood, cell free genomic material (e.g., cell-free DNA) is utilized to detect sex chromosome copy number variants in the fetus.

In a separate aspect, the invention provides assay systems and methods that utilize detection of selected sequences within the PARs for determining sex chromosome copy number variants in the fetus from a maternal biological sample, e.g., maternal blood, plasma and serum, without determining the gender of the fetus.

Thus, the invention provides an assay system for determination of fetal sex in a maternal sample, comprising providing a maternal sample comprising maternal and fetal cell free DNA, amplifying two or more selected nucleic acid regions from a pseudoautosomal region of a sex chromosome in the maternal sample, amplifying two or more selected nucleic acid regions from an X chromosome outside the pseudoautosomal regions, determining the relative frequency of the selected nucleic acid regions from the sex chromosomes in the maternal sample, comparing the relative frequency of the selected nucleic acid regions, and identifying the fetal sex based on the compared relative frequencies of the selected nucleic acid regions.

In some aspects of this embodiment of the invention, the chromosome dosage of the first and second chromosome is estimated by interrogating one or more loci on two or more chromosomes in both the fetus and mother. In some aspects, the chromosome dosage of the first and second fetal chromosome is estimated by interrogating at least ten, at least twenty, at least twenty-four, at least forty-eight, at least ninety-six, at least one hundred, at least one hundred fifty, at least one hundred ninety two, or at least three hundred eighty four.

at least two hundred, or at least four hundred or more loci on each chromosome for which chromosome dosage is being estimated. In some aspects of this embodiment, the loci interrogated for estimation of dosage of the first and second fetal chromosome are non-polymorphic loci.

In other aspects of this embodiment, the fetal nucleic acid proportion is determined by interrogating one or more polymorphic loci in both the fetus and the mother. In some aspects, the fetal nucleic acid proportion in the maternal sample is performed by interrogating at least ten, at least twenty, at least twenty-five, at least forty-eight, at least ninety-six, at least one hundred, at least one hundred fifty, or at least two hundred or more polymorphic loci.

In some aspects of this embodiment of the invention the odds ratio reflects the likelihood of a chromosome dosage abnormality for the first fetal chromosome based on a value of the likelihood of the chromosome being trisomic and the value of likelihood of the chromosome being disomic; and in yet other aspects of this embodiment, the odds ratio reflects the likelihood of a chromosome dosage abnormality for the first fetal chromosome based on a value of the likelihood of the chromosome being monosomic and the value of the likelihood of the chromosome being disomic.

A specific embodiment of the present invention provides a computer-implemented process to calculate a risk of a fetal sex chromosome aneuploidy in a maternal sample comprising: estimating the fetal sex chromosome dosage in the maternal sample by interrogating loci from the PAR; estimating the fetal chromosome dosage for one or more reference chromosomes in the maternal sample; determining a fetal nucleic acid proportion in the maternal sample; calculating a value of the likelihood of a fetal sex chromosome aneuploidy by comparing the chromosome dosage of the fetal sex chromosomes to the chromosome dosage of one or more reference fetal chromosomes in view of the fetal nucleic acid proportion in the maternal sample; calculating a value of the likelihood that the sex chromosomes are disomic by comparing the chromosome dosage of the fetal sex chromosome to the chromosome dosage of the one or more reference chromosomes in view of the fetal nucleic acid proportion in the maternal sample; computing a value of the probability of a chromosome dosage abnormality for the fetal sex chromosomes based on a value of the likelihood of the sex chromosomes being aneuploid and the value of the likelihood of the sex chromosomes being disomic; and adjusting the computed odds ratio using information related to one or more extrinsic factors.

These and other aspects will be described in more detail herein.

### DESCRIPTION OF THE FIGURES

FIG. 1 is a block diagram illustrating an exemplary system environment.

### DETAILED DESCRIPTION OF THE INVENTION

The methods described herein may employ, unless otherwise indicated, conventional techniques and descriptions of molecular biology (including recombinant techniques), cell biology, biochemistry, and microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W. H. Freeman, New York (1995); Gait, “Oligonucleotide Synthesis: A Practical Approach” IRL Press, London (1984); Nelson and Cox, Lehninger, Principles of Biochemistry, 3^{rd} Ed., W. H. Freeman Pub., New York (2000); and Berg et al., Biochemistry, 5^{th} Ed., W. H. Freeman Pub., New York (2002), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by appended claims.

It should be noted that as used herein and in the appended claims, the singular forms “a,” “and,” and “the” include plural referents unless the context clearly dictates otherwise. Thus, for example, reference to “a nucleic acid region” refers to one, more than one, or mixtures of such regions, and reference to “an assay” includes reference to equivalent steps and methods known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those included limits are also included in the invention.

All publications mentioned herein are incorporated by reference for the purpose of describing and disclosing the formulations and methodologies that are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “amplified nucleic acid” is any nucleic acid molecule whose amount has been increased at least two fold by any nucleic acid amplification or replication method performed in vitro as compared to its starting amount in a maternal sample.

The term “autosomal region” refers to any region of chromosomes 1-22.

The term “chromosomal abnormality” refers to any genetic variant for all or part of a chromosome. The genetic variants may include but not be limited to any copy number variant such as duplications or deletions, translocations, inversions, and mutations. The term chromosomal abnormality as used herein particularly refers to an abnormal number of sex chromosomes or a region thereof, e.g., an abnormal number of regions on PAR1 and PAR2 alone or in comparison with other regions on the X chromosome or Y chromosome.

The terms “complementary” or “complementarity” are used in reference to nucleic acid molecules (i.e., a sequence of nucleotides) that are related by base-pairing rules. Complementary nucleotides are, generally, A and T (or A and U), or C and G. Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100% complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions. Stringent hybridization conditions will typically include salt concentrations of less than about 1 M, more usually less than about 500 mM and preferably less than about 200 mM. Hybridization temperatures are generally at least about 2° C. to about 6° C. lower than melting temperatures (Tₘ).

The term “correction index” refers to an index that may contain additional nucleotides that allow for identification and correction of amplification, sequencing or other experimental errors including the detection of deletion, substitution, or insertion of one or more bases during sequencing as well as nucleotide changes that may occur outside of sequencing such as oligo synthesis, amplification, and any other aspect of the assay. These correction indices may be stand-alone indices that are separate sequences, or they may be embedded within other indices to assist in confirming accuracy of the experimental techniques used, e.g., a correction index may be a subset of sequences of a locus index or an identification index.

The term “diagnostic tool” as used herein refers to any composition or assay of the invention used in combination as, for example, in a system in order to carry out a diagnostic test or assay on a patient sample.

The term “disomic” when referring to the sex chromosomes can mean either an XX or an XY genotype.

The term “hybridization” generally means the reaction by which the pairing of complementary strands of nucleic acid occurs. DNA is usually double-stranded, and when the strands are separated they will re-hybridize under the appropriate conditions. Hybrids can form between DNA-DNA, DNA-RNA or RNA-RNA. They can form between a short strand and a long strand containing a region complementary to the short one. Imperfect hybrids can also form, but the more imperfect they are, the less stable they will be (and the less likely to form).

The term “identification index” refers generally to a series of nucleotides incorporated into a primer region of an amplification process for unique identification of an amplification product of a nucleic acid region. Identification index sequences are preferably 6 or more nucleotides in length.

In a preferred aspect, the identification index is long enough to have statistical probability of labeling each molecule with a target sequence uniquely. For example, if there are 3000 copies of a particular target sequence, there are substantially more than 3000 identification indexes such that each copy of a particular target sequence is likely to be labeled with a unique identification index. The identification index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay. The index may be combined with any other index to create one index that provides information for two properties (e.g., sample-identification index, locus-identification index).

The terms “locus” and “loci” as used herein refer to a nucleic acid region of known location in a genome.

The term “locus index” refers generally to a series of nucleotides that correspond to a known locus on a chromosome. Generally, the locus index is long enough to label each known locus region uniquely. For instance, if the method uses 192 known locus regions corresponding to 192 individual sequences associated with the known loci, there are at least 192 unique locus indexes, each uniquely identifying a region indicative of a particular locus on a chromosome. The locus indices used in the methods of the invention may be indicative of different loci on a single chromosome as well as known loci present on different chromosomes within a sample. The locus index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay.

The term “maternal sample” as used herein refers to any sample taken from a pregnant mammal which comprises both fetal and maternal cell free genomic material (e.g., DNA). Preferably, maternal samples for use in the invention are obtained through relatively non-invasive means, e.g., phlebotomy or other standard techniques for extracting peripheral samples from a subject.

The term “melting temperature” or Tₘ is commonly defined as the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+16.6(log 10[Na+])0.41(%[G+C])−675/n−1.0 m, when a nucleic acid is in aqueous solution having cation concentrations of 0.5 M or less, the (G+C) content is between 30% and 70%, n is the number of bases, and m is the percentage of base pair mismatches (see, e.g., Sambrook J et al., Molecular Cloning, A Laboratory Manual, 3rd Ed., Cold Spring Harbor Laboratory Press (2001)). Other references include more sophisticated computations, which take structural as well as sequence characteristics into account for the calculation of Tₘ.

“Microarray” or “array” refers to a solid phase support having a surface, preferably but not exclusively a planar or substantially planar surface, which carries an array of sites containing nucleic acids such that each site of the array comprises substantially identical or identical copies of oligonucleotides or polynucleotides and is spatially defined and not overlapping with other member sites of the array; that is, the sites are spatially discrete. The array or microarray can also comprise a non-planar interrogatable structure with a surface such as a bead or a well. The oligonucleotides or polynucleotides of the array may be covalently bound to the solid support, or may be non-covalently bound. Conventional microarray technology is reviewed in, e.g., Schena, Ed., Microarrays: A Practical Approach, IRL Press, Oxford (2000). “Array analysis”, “analysis by array” or “analysis by microarray” refers to analysis, such as, e.g., sequence analysis, of one or more biological molecules using a microarray.

The term “mixed sample” as used herein refers to any sample comprising cell free genomic material (e.g., DNA) from two or more cell types of interest. Exemplary mixed samples include a maternal sample (e.g., maternal blood, serum or plasma comprising both maternal and fetal DNA), and a peripherally-derived somatic sample (e.g., blood, serum or plasma comprising different cell types, e.g., hematopoietic cells, mesenchymal cells, and circulating cells from other organ systems). Mixed samples include samples with genomic material from two different sources comprising cells that are from two different individuals, e.g., a sample with both maternal and fetal genomic material or a sample from a transplant patient that comprises cells from both the donor and recipient.

The term “monsomic” when referring to the sex chromosomes means an XO genotype, i.e. one copy of the X chromosome and no copy of the Y chromosome.

By “non-polymorphic”, when used with respect to detection of selected nucleic acid regions, is meant a detection of such nucleic acid region, which may contain one or more polymorphisms, but in which the detection is not reliant on detection of the specific polymorphism within the region. Thus a selected nucleic acid region may contain a polymorphism, but detection of the region using the assay system of the invention is based on occurrence of the region rather than the presence or absence of a particular polymorphism in that region.

The tern “non-PAR” refers to a region on the X or Y chromosome that is outside the pseudoautosomal region.

As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP.

According to the present invention, a “nucleotide” may be unlabeled or detectably labeled by well known techniques. Fluorescent labels and their attachment to oligonucleotides are described in many reviews, including Haugland, Handbook of Fluorescent Probes and Research Chemicals, 9th Ed., Molecular Probes, Inc., Eugene Oreg. (2002); Keller and Manak, DNA Probes, 2nd Ed., Stockton Press, New York (1993); Eckstein, Ed., Oligonucleotides and Analogues: A Practical Approach, IRL Press, Oxford (1991); Wetmur, Critical Reviews in Biochemistry and Molecular Biology, 26:227-259 (1991); and the like. Other methodologies applicable to the invention are disclosed in the following sample of references: Fung et al., U.S. Pat. No. 4,757,141; Hobbs, Jr., et al., U.S. Pat. No. 5,151,507; Cruickshank, U.S. Pat. No. 5,091,519; Menchen et al., U.S. Pat. No. 5,188,934; Begot et al., U.S. Pat. No. 5,366,860; Lee et al., U.S. Pat. No. 5,847,162; Khanna et al., U.S. Pat. No. 4,318,846; Lee et al., U.S. Pat. No. 5,800,996; Lee et al., U.S. Pat. No. 5,066,580: Mathies et al., U.S. Pat. No. 5,688,648; and the like. Labeling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; and 2003/0017264. Detectable labels include, for example, radioactive isotopes, fluorescent labels, chemiluminescent labels, bioluminescent labels and enzyme labels.

Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′ dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110] dCTP, [R6G]dCTP, [TAMRA]dCTP, [JOE] ddATP, [R6G]ddATP, [FAM]ddCTP, [R110] ddCTP, [TAMRA]ddGTP, [ROX] ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg.

The terms “oligonucleotides” or “oligos” as used herein refer to linear oligomers of natural or modified nucleic acid monomers, including deoxyribonucleotides, ribonucleotides, anomeric forms thereof, peptide nucleic acid monomers (PNAs), locked nucleotide acid monomers (LNA), and the like, or a combination thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200 or more. Suitable nucleic acid molecules may be prepared by the phosphoramidite method described by Beaucage and Carruthers (Tetrahedron Lett., 22:1859-1862 (1981)), or by the triester method according to Matteucci, et al. (J. Am. Chem. Soc., 103:3185 (1981)), both incorporated herein by reference, or by other chemical methods such as using a commercial automated oligonucleotide synthesizer.

The term “pseudoautosomal regions” refers to the regions on chromosomes X and Y that display autosomal inheritance patterns.

As used herein the term “polymerase” refers to an enzyme that links individual nucleotides together into a long strand, using another strand as a template. There are two general types of polymerase—DNA polymerases, which synthesize DNA, and RNA polymerases, which synthesize RNA. Within these two classes, there are numerous sub-types of polymerases, depending on what type of nucleic acid can function as template and what type of nucleic acid is formed.

As used herein “polymerase chain reaction” or “PCR” refers to a technique for replicating a specific piece of target DNA in vitro, even in the presence of excess non-specific DNA. Primers are added to the target DNA, where the primers initiate the copying of the target DNA using nucleotides and, typically, Taq polymerase or the like. By cycling the temperature, the target DNA is repetitively denatured and copied. A single copy of the target DNA, even if mixed in with other, random DNA, can be amplified to obtain billions of replicates. The polymerase chain reaction can be used to detect and measure very small amounts of DNA and to create customized pieces of DNA. In some instances, linear amplification methods may be used as an alternative to PCR.

The term “polymorphism” as used herein refers to any genetic changes in a loci that may be indicative of that particular loci, including but not limited to single nucleotide polymorphisms (SNPs), methylation differences, short tandem repeats (STRs), and the like.

Generally, a “primer” is an oligonucleotide used to, e.g., prime DNA extension, ligation and/or synthesis, such as in the synthesis step of the polymerase chain reaction or in the primer extension techniques used in certain sequencing reactions. A primer may also be used in hybridization techniques as a means to provide complementarity of a nucleic acid region to a capture oligonucleotide for detection of a specific nucleic acid region.

The term “research tool” as used herein refers to any composition or assay of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The term “sample index” refers generally to a series of unique nucleotides (i.e., each sample index is unique to a sample in a multiplexed assay system for analysis of multiple samples). The sample index can thus be used to assist in nucleic acid region identification for multiplexing of different samples in a single reaction vessel, such that each sample can be identified based on its sample index. In a preferred aspect, there is a unique sample index for each sample in a set of samples, and the samples are pooled during sequencing. For example, if twelve samples are pooled into a single sequencing reaction, there are at least twelve unique sample indexes such that each sample is labeled uniquely. The index may be combined with any other index to create one index that provides information for two properties (e.g., sample-identification index, sample-locus index).

The term “selected nucleic acid region” as used herein refers to a nucleic acid region corresponding to an individual chromosome. Such selected nucleic acid regions may be directly isolated and enriched from the sample for detection, e.g., based on hybridization and/or other sequence-based techniques, or they may be amplified using the sample as a template prior to detection of the sequence. Nucleic acids regions for use in the assay systems of the present invention may be selected on the basis of DNA level variation between individuals, based upon specificity for a particular chromosome, based on CG content and/or required amplification conditions of the selected nucleic acid regions, or other characteristics that will be apparent to one skilled in the art upon reading the present disclosure.

The terms “sequencing”, “sequence determination” and the like as used herein refers generally to any and all biochemical methods that may be used to determine the order of nucleotide bases in a nucleic acid.

The term “specifically binds”, “specific binding” and the like as used herein, when referring to a binding partner (e.g., a nucleic acid probe or primer, antibody, etc.) that results in the generation of a statistically significant positive signal under the designated assay conditions. Typically the interaction will subsequently result in a detectable signal that is at least twice the standard deviation of any signal generated as a result of undesired interactions (background).

The term “trisomic” when referring to the sex chromosomes can mean an XXX, XXY or XYY genotype.

### The Invention in General

Pseudoautosomal regions (PARs) are homologous sequences found on both the X and Y chromosome. The present invention provides assay systems that utilize these regions to detect the number of sex chromosomes in a mixed sample, allowing both sex determination of two or more cell populations within a mixed sample and detection of genetic abnormalities within two or more cell populations within a mixed sample. This can be particularly useful in maternal samples to determine the sex of a fetus and/or any sex chromosome abnormalities in a fetus. These assays also provide detection of sex-mismatched cells in an individual, e.g., due to the presence of cells resulting from a sex-mismatched transplantation.

In one embodiment the detection methods of the invention are not reliant upon the presence or absence of any polymorphic or mutation information in the PARs and/or non-PARs, and thus are conceptually agnostic as to the genetic variation that may be present in any chromosomal region under interrogation. In another embodiment the detection methods of the invention rely upon the presence or absence of polymorphic information in the PAR and/or non-PAR. Both such methods, as well as combinations thereof, are useful for any mixed sample containing cell free genomic material (e.g., DNA) from two or more cell types of interest, e.g., mixed samples comprising maternal and fetal cell free DNA and mixed samples comprising cell free DNA from a transplant donor and recipient, and the like.

The assay methods of the invention provide a selected enrichment of nucleic acid regions for copy number variant detection of the PARs or other selected regions on the sex chromosomes. A distinct advantage of the invention is that the enriched selected nucleic acid regions can be further analyzed using a variety of detection and quantification techniques, including but not limited to hybridization techniques, digital PCR and high throughput sequencing determination techniques. Selection probes can be designed against any number of nucleic acid regions on the sex chromosome. Although amplification prior to the identification and quantification of the selection nucleic acids regions is not mandatory, limited amplification prior to detection is preferred.

The present invention provides an improved system over more random techniques which have been used by others to detect copy number variations in mixed samples such as maternal blood. These aforementioned approaches rely upon sequencing of a statistically significant population of DNA fragments in a sample, followed by mapping of these fragments or otherwise associating the fragments to their appropriate chromosomes. The identified fragments are then compared against each other or against some other reference (e.g., normal chromosomal makeup) to determine copy number variation of sex chromosomes. These methods are inherently inefficient from the present invention, as the sex chromosomes only constitute a minority of data that is generated from the detection of such DNA fragments in the mixed samples.

Techniques that are dependent upon a very broad sampling of DNA in a sample are providing a very broad coverage of the DNA analyzed, but in fact are sampling the DNA contained within a sample on a 1× or less basis (i.e., subsampling). In contrast, the selective amplification and/or enrichment used in the present assays are specifically designed to provide depth of coverage of particular nucleic acids of interest on the sex chromosomes, and provide a “super-sampling” of such selected regions with an average sequence coverage of preferably 2× or more, more preferably sequence coverage of 100× of more, even more preferably sequence coverage of 1000× or more of the selected nucleic acids present in the initial mixed sample.

The methods of the invention provide a more efficient and economical use of data, and the substantial majority of sequences analyzed following sample amplification result in affirmative information about the presence of a particular chromosome in the sample. Thus, unlike techniques relying on massively parallel sequencing or random digital “counting” of chromosome regions and subsequent identification of relevant data from such counts, the assay system of the invention provides a much more efficient use of data collection than the random approaches taught by others in the art.

The sequences analyzed using the assay system of the present invention are enriched and/or amplified representative sequences selected from various regions of the sex chromosomes to determine the relative quantity of the sex chromosomes in the mixed sample, and the substantial majority of sequences analyzed are informative of the presence of a region on a sex chromosome that is useful in sex determination and/or aneuploidy detection. These techniques do not require the analysis of large numbers of sequences which are not from the sex chromosomes and which do not provide information on the relative quantity of the sex chromosomes.

### Detection of Sex Chromosome Aneuploidies

The present invention provides methods for identifying fetal chromosomal aneuploidies in maternal samples comprising both maternal and fetal DNA. This can be performed using enrichment and/or amplification methods for identification of nucleic acid regions corresponding to specific sex chromosomes and/or reference chromosomes in the maternal sample.

In one aspect, this invention utilizes the analysis of pseudoautosomal regions to determine whether an abnormal number of sex chromosomes are present in one or more cell populations within a maternal sample. In the case of a normal male or female, two PARs will be present. In the case of monosomy such as Turner syndrome (XO), only one of the PARs will be present. In cases of trisomy such as Klinefelter's syndrome (XXY), triple X syndrome (XXX), or XYY, three PARs will be present. Identification of these aneuploidies can be detected through the identification of an abnormal PAR ratio in a mixed sample in comparison to selected regions from one or more autosomes and/or predicted levels of PAR sequences. The detection of a copy number variant in the sex chromosomes can also utilize a comparison to a reference genomic region from an autosome or a non-PAR region of a sex chromosome.

The detection of selected regions of the PARs and autosomes in a mixed sample can be used to determine aneuploidy by determining the ratios of the selected PAR loci with the autosomal loci. In certain aspects, selected regions of the PARs and autosomes in a maternal sample can be used to determine aneuploidy in a fetus by determining the ratios of the selected PAR loci with the autosomal loci in the maternal sample. In other aspects, selected regions of the PARs and non-PAR regions of sex chromosomes in a maternal sample can be used to determine aneuploidy in a fetus by determining the ratios of the selected PAR loci with the non-PAR sex chromosome loci in the maternal sample. Although knowledge of percent fetal DNA is not required for determination of aneuploidy, in certain aspects, the ratios are determinative based on the ratios in view of the percent fetal DNA in the sample. Tables 1 and 2 illustrate exemplary ratios for different genotypes when the amount of fetal DNA in a maternal sample is 10%.

TABLE 2
Relative Ratios for Sex Chromosomal Frequencies
Compared to Autosome Frequencies

| | XX | XY | XXX | XXY | XYY | XO |
|---|---|---|---|---|---|---|
| X non-PAR | 1000:1000 | 950:1000 | 1050:1000 | 1000:1000 | 950:1000 | 950:1000 |
| Regions to | | | | | | |
| Autosomes | | | | | | |
| Y non-PAR | 0:1000 | 50:1000 | 0:1000 | 50:1000 | 100:1000 | 0:1000 |
| Regions to | | | | | | |
| Autosomes | | | | | | |
| PARs to | 1000:1000 | 1000:1000 | 1050:1000 | 1050:1000 | 1050:1000 | 950:1000 |
| Autosomes | | | | | | |

TABLE 2
Relative Ratios for Sex Chromosomal Frequencies Compared to PAR Frequencies

| | XX | XY | XXX | XXY | XYY | XO |
|---|---|---|---|---|---|---|
| X non-PAR | 1000:1000 | 950:1000 | 1050:1050 | 1000:1050 | 950:1050 | 950:950 |
| Regions:PARs | | | | | | |
| Y non-PAR | 0:1000 | 50:1000 | 0:1050 | 50:1050 | 100:1050 | 0:950 |
| Regions:PARS | | | | | | |

It should be noted that in the cases of sex chromosome trisomy, three PARs will be present but given the regions are homologous, it would be challenging to determine what specific sex chromosome has been duplicated without determination of additional sequences from the X and/or Y chromosome. In certain instances, this may be preferable since it does not require determination of sex or determination of non-PAR Y sequence In other certain aspects, additional analysis of sequences from the non-PAR region of the X or Y chromosome can be used to determine which specific sex chromosome trisomy is present. In the instance where non-PAR regions of X or Y sequences are detected, this is preferably performed in the same reaction and/or vessel as the other interrogations, although of course it could be performed as a separate reaction. In a preferred embodiment, only the non-PAR region of X is detected to determine the specific sex chromosome trisomy present.

The detection of PAR sequences can be used alone or in conjunction with other methods of sex determination or aneuploidy, e.g., ultrasound techniques.

In a preferred aspect, PARs are used to detect sex chromosome copy number variants in the fetus from a maternal biological sample. In the case where the maternal biological sample is blood, cell free genomic material (e.g., DNA) could be evaluated for copy number variants in the fetus. The methods use counting of selected cell free DNA fragments and determining fetal aneuploidy from over- or under-representation of the sex chromosomes. The analysis of PARs using targeted analysis of genomic fragments is performed such as that described in U.S. 61/436,132 and U.S. 61/436,135. The determination of PARs disomy versus trisomy or monosomy may use incorporate the percent fetal such as that described in U.S. Ser. Nos. 13/316,154 and 13/338,963. The determination of PARs disomy versus trisomy or monosomy may use a Z-score cut-off such as described in U.S. Ser Nos. 13/013,732, 13/205,570 and 13/205,603.

### Sex Determination

In another aspect, the invention provides assay systems to determine the sex of one or more normal fetus by combining analysis of PAR and non-PAR regions in one or both of the sex chromosomes. Analysis of PARs allows the determination whether disomy is present or not in the sex chromosomes. However, in situations where PARs suggest sex chromosome disomy, distinguishing between XX and XY is not possible. This can be overcome by performing additional analyses of non-PARs. In a preferred embodiment, the non-PARs are on chromosome X. Analysis of selected loci from the non-pseudoautosomal regions of X in comparison to one or more automosmal regions that suggests only one copy of chromosome X in a disomic fetus suggests a male fetus, while analysis of selected regions of the X chromosome in comparison to one or more automosmal regions that suggests only two copies of X in a disomic fetus suggests a girl. Thus, if analysis of the PARs in comparison to one or more autosomal regions suggests sex chromosome disomy, and analysis of X specific regions suggest X disomy, then the fetus is likely female. If analysis of PARs in comparison to one or more autosomal regions suggests sex chromosome disomy and analysis of the non-PARs X chromosome suggests only one X chromosome in the fetus, the fetus is likely a male. If analysis of PARs in comparison to one or more autosomal regions suggests sex chromosome disomy, and copies of a Y specific region are detected, this would suggest the fetus is a male.

In a specific aspect, a threshold level is used (e.g., based on a Z-score) to determine whether the fetus is likely to has a sex chromosome monosomy or trisomy. For example, analysis of a maternal sample resulting in a Z score that is at least 3 times greater than the chromosomal variations (CVs) seen in maternal samples with a fetus disomic for the sex chromosomes would indicate that the fetus has a sex chromosome trisomy. Likewise, analysis of a maternal sample resulting in a Z score that is at least 3 times below the CVs in maternal samples with a fetus disomic for the sex chromosomes would indicate that the fetus has a sex chromosome monosomy. In other aspects, the chromosomal ratio of the sex chromosomes and one or more autosomes is compared to the mean chromosomal ratio of the sex chromosomes and one or more autosomes from a reference population of maternal samples having fetuses with sex chromosome disomy, and the threshold for identifying the presence or absence of an aneuploidy is at least three times the chromosomal variation in of the reference population.

This analysis can also use ratios of maternal and fetal DNA to determine the likely sex of multiple fetus in utero using mathematical ratios of the sex chromosomes and detection of X and Y.

### Determination of the Specific Type of Sex Chromosome Aneuploidies

In another aspect, the invention provides assay systems to determine the specific type of sex chromosome aneuploidy. Analysis of PARs allows the determination whether an aneuploidy (e.g., triploidy, tetraploidy, etc.) is present or not in the sex chromosomes. However, in situations where PARs suggest sex chromosome trisomy, distinguishing between XXX, XXY, XYY is not possible. This can be overcome by performing additional analyses of a sex chromosomal region outside of the pseudoautomosomal regions (non-PARs). In a preferred aspect, the non-PARs are on chromosome X. The number of copies of non-PARs in the fetus may be determined by comparing the non-PARs to one or more autosomes with a likelihood determination for one, two or three copies through the use of percent fetal such as described in U.S. Ser. Nos. 13/316,154 and 13/338,963 or through the use of a Z-score cut-off such as described in U.S. 13/013,732, 13/205,570 and 13/205,603.

Analysis of PARs in comparison to one or more autosomal regions suggests sex chromosome trisomy in the fetus and analysis of the non-PARs on chromosome X also suggests three copies for chromosome X in the fetus, strongly suggest a XXX trisomy in the fetus. Analysis of PARs in comparison to one or more autosomal regions suggests sex chromosome trisomy in the fetus and analysis of the non-PARs in comparison to one or more autosomal regions suggests two copies of X chromosome in the fetus strongly suggest a XXY trisomy in the fetus. Analysis of PARs in comparison to one or more autosomal regions suggests sex chromosome trisomy in the fetus and analysis of non-PARs of the X chromosome in comparison to one or more autosomal regions suggests one copy of X chromosome in the fetus strongly suggest a XYY trisomy. In another preferred aspect, the non-PARs are on chromosome Y. In this aspect, the non-PARs on chromosome Y are compared to one or more autosomal regions to determine whether there is zero, one or two copies of the Y chromosome using a likelihood determined by the use of percent fetal or a Z-score cutoff such as described in U.S. Ser Nos. 13/316,154 and 13/338,963. Analysis of PARs in comparison to one or more autosomal regions suggests sex chromosome trisomy in the fetus and analysis of non-PARs of the chromosome Y in comparison to one or more autosomal regions suggests no Y chromosome in the fetus suggest a XXX trisomy in the fetus. Analysis of PARs in comparison to one or more autosomal regions suggests sex chromosome trisomy in the fetus and analysis of non-PARs of the chromosome Y in comparison to one or more autosomal regions suggests one Y chromosome in the fetus strongly suggest a XXY trisomy. If analysis of PARs in comparison to one or more autosomal regions suggests sex chromosome trisomy in the fetus and analysis of non-PARs of the chromosome Y in comparison to one or more autosomal regions suggests two Y chromosomes in the fetus strongly suggest a XYY trisomy.

### Assay System Detection

The assay systems utilize nucleic acid probes designed to identify, and preferably to isolate, PARs or other selected nucleic acids regions in a mixed sample that correspond to individual sex chromosomes. These probes are specifically designed to hybridize to a selected nucleic acid region of a sex chromosome, and thus quantification of the nucleic acid regions in a mixed sample using these probes is indicative of the copy number of a particular sex chromosome in the mixed sample.

In preferred aspects, the assay systems of the invention employ one or more selective amplification or enrichment steps (e.g., using one or more primers that specifically hybridize to a selected nucleic acid region) to enhance the DNA content of a sample and/or to provide improved mechanisms for isolating, amplifying or analyzing the selected nucleic acid regions. This is in direct contrast to the random amplification approach used by others employing, e.g., massively parallel sequencing, as such amplification techniques generally involve random amplification of all or a substantial portion of the genome.

In a general aspect, the user of the invention analyzes multiple target sequences on different chromosomes and determines the frequency or amount of the target sequences of the chromosomes together. When multiple target sequences are analyzed on the sex chromosomes, a preferred embodiment is to amplify all of the target sequences for each sample in one reaction vessel. The frequency or amount of the multiple target sequences on the different sex chromosomes is then compared to the frequency or amount of the multiple target sequences on autosomal chromosomes to determine whether a chromosomal abnormality exists.

In one aspect, the user of the invention analyzes multiple target sequences on multiple chromosomes and averages the frequency of the target sequences on the multiple chromosomes together. Normalization or standardization of the frequencies can be performed for one or more target sequences.

In one aspect, the number of multiple target sequences in the PAR, the non-PAR regions of X and the autosomal regions is each at least 20. In one aspect, the number of multiple target sequences in the PAR, the non-PAR regions of X and the autosomal regions is each at least 24. In one aspect, the number of multiple target sequences in the PAR, the non-PAR regions of X and the autosomal regions is each at least 48. In one aspect, the number of multiple target sequences in the PAR, the non-PAR regions of X and the autosomal regions is each at least 96. In one aspect, the number of multiple target sequences in the PAR, the non-PAR regions of X and the autosomal regions is each at least 192. In one aspect, the number of multiple target sequences in the PAR, the non-PAR regions of X and the autosomal regions is each at least 288. In one aspect, the number of multiple target sequences in the PAR, the non-PAR regions of X and the autosomal regions is each at least 384.

In another aspect, the user of the invention sums the frequencies of the target sequences on the sex chromosome and then compares the sum of the target sequences on the sex chromosome against an autosome to determine whether a chromosomal abnormality exists. In another aspect, one analyzes subsets of target sequences on each sex chromosome to determine whether a chromosomal abnormality exists. The comparison can be made either within the same or different chromosomes.

In certain aspects, the data used to determine the frequency of the target sequences may exclude outlier data that appear to be due to experimental error, or that have elevated or depressed levels based on an idiopathic genetic bias within a particular sample. In one example, the data used for summation may exclude DNA regions with a particularly elevated frequency in one or more samples. In another example, the data used for summation may exclude target sequences that are found in a particularly low abundance in one or more samples.

In another aspect subsets of loci can be chosen randomly within the PARs and other regions of the sex chromosomes but with sufficient numbers of loci to yield a statistically significant result in determining whether a sex chromosomal abnormality exists or to ensure accuracy of sex determination. Multiple analyses of different subsets of loci can be performed within a mixed sample to yield more statistical power. For example, if there are 100 selected regions for chromosome 21 and 100 selected regions for chromosome 18, a series of analyses could be performed that evaluate fewer than 100 regions for each of the chromosomes. In this example, target sequences are not being selectively excluded.

The quantity of different nucleic acids detectable on certain chromosomes may vary depending upon a number of factors, including general representation of fetal loci in maternal samples, degradation rates of the different nucleic acids representing fetal loci in maternal samples, sample preparation methods, and the like. Thus, in another aspect, the quantity of particular loci on a chromosome is summed to determine the loci quantity for different chromosomes in the sample. The loci frequency is summed for a particular chromosome, and the sum of the loci are used to determine aneuploidy. This aspect of the invention sums the frequencies of the individual loci on each chromosome and then compares the sum of the loci on one chromosome (e.g., Y) against another chromosome (e.g., X or an autosome) to determine whether a chromosomal difference exists.

The nucleic acids analyzed using the assay systems of the invention are preferably selectively amplified and optionally isolated from the mixed sample using primers specific to the nucleic acid region of interest (e.g., to a locus of interest in a maternal sample). The primers for such selective amplification are designed to isolate regions may be chosen for various reasons, but are preferably designed to 1) efficiently amplify a region, e.g., from a selected locus in a PAR; 2) have a predictable range of expression from the sources in different mixed samples; 3) be distinctive to the particular chromosome or chromosomal region, i.e., not amplify homologous regions on other chromosomes or chromosomal regions. The following are exemplary techniques that may be employed in the assay system of the invention.

### Selected Enrichment and Amplification

Numerous selective amplification methods can be used to provide the amplified nucleic acids that are analyzed in the assay systems of the invention, and such methods are preferably used to increase the copy numbers of a nucleic acid region of interest in a mixed sample in a manner that allows preservation of information concerning the initial content of the nucleic acid region in the mixed sample. Although not all combinations of amplification and analysis are described herein in detail, it is well within the skill of those in the art to utilize different amplification methods and/or analytic tools to isolate and/or analyze the nucleic acids of region consistent with this specification, and such variations will be apparent to one skilled in the art upon reading the present disclosure.

Such amplification methods include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al. 1992, Nucleic Acids Res. 20(7):1691-6, 1992, and rolling circle amplification, described in U.S. Pat. No. 5,648,245. Other amplification methods that may be used are described in, U.S. Pat. Nos. 5,242,794, 5,494,810, 4,988,617 and in U.S. Ser. No. 09/854,317 and U.S. Pub. No. 20030143599, each of which is incorporated herein by reference. In some aspects DNA is amplified by multiplex locus-specific PCR. In a preferred aspect the DNA is amplified using adaptor-ligation and single primer PCR. Other available methods of amplification, such as balanced PCR (Makrigiorgos, et al. (2002), Nat Biotechnol, Vol. 20, pp. 936-9) and isothermal amplification methods such as nucleic acid sequence based amplification (NASBA) and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a nucleic acid region of interest. Such primers may be used to amplify DNA of any length so long that it contains the nucleic acid region of interest in its sequence.

The length of an amplified selected nucleic acid from a genomic region of interest is generally long enough to provide enough sequence information to distinguish it from other nucleic acids that are amplified and/or selected. Generally, an amplified nucleic acid is at least about 16 nucleotides in length, and more typically, an amplified nucleic acid is at least about 20 nucleotides in length. In a preferred aspect of the invention, an amplified nucleic acid is at least about 30 nucleotides in length. In a more preferred aspect of the invention, an amplified nucleic acid is at least about 32, 40, 45, 50, or 60 nucleotides in length. In other aspects of the invention, an amplified nucleic acid can be about 100, 150 or up to 200 in length.

In certain aspects, the selected amplification comprises an initial linear amplification step. This can be particularly useful if the starting amount of DNA is quite limited, e.g., where the cell-free DNA in a sample is available in limited quantities. This mechanism increases the amount of DNA molecules that are representative of the original DNA content, and help to reduce sampling error where accurate quantification of the DNA or a fraction of the DNA (e.g., fetal DNA contribution in a maternal sample) is needed.

Thus, in one aspect, a limited number of cycles of sequence-specific linear amplification are performed on the starting maternal sample comprising cell free DNA. The number of cycles is generally less than that used for a typical PCR amplification, e.g., 5-30 cycles or fewer. Primers or probes may be designed to amplify specific genomic segments or regions. The primers or probes may be modified with an end label at the 5′ end (e.g., with biotin) or elsewhere along the primer or probe such that the amplification products could be purified or attached to a solid substrate (e.g., bead or array) for further isolation or analysis. In a preferred aspect, the primers are multiplexed such that a single reaction yields multiple DNA fragments from different regions. Amplification products from the linear amplification could then be further amplified with standard PCR methods or with additional linear amplification.

For example, cell free DNA can be isolated from blood, plasma, or serum from a pregnant woman, and incubated with primers against a set number of nucleic acid regions that correspond to the sex chromosomes. Preferably, the number of primers used for initial linear amplification will be 12 or more, more preferably 24 or more, more preferably 36 or more, even more preferably 48 or more, and even more preferably 96 or more. Each of the primers corresponds to a single nucleic acid region, and is optionally tagged for identification and/or isolation. A limited number of cycles, preferably 10 or fewer, are performed with linear amplification. The amplification products are subsequently isolated, e.g., when the primers are linked to a biotin molecule the amplification products can be isolated via binding to avidin or streptavidin on a solid substrate. The products are then subjected to further biochemical processes such as further amplification with other primers and/or detection techniques such as sequence determination and hybridization.

Efficiencies of linear amplification may vary between sites and between cycles so that in certain systems normalization may be used to ensure that the products from the linear amplification are representative of the nucleic acid content starting material. One practicing the assay system of the invention can utilize information from various samples to determine variation in nucleic acid levels, including variation in different nucleic acid regions in individual samples and/or between the same nucleic acid regions in different samples following the limited initial linear amplification. Such information can be used in normalization to prevent skewing of initial levels of DNA content.

### Universal Amplification

In preferred aspects of the invention, the selectively detected nucleic acid regions are preferably amplified following selective amplification or enrichment, either prior to or during the nucleic acid region detection techniques. In another aspect of the invention, nucleic acid regions are selectively amplified during the nucleic acid region detection technique without any prior amplification. In a multiplexed assay system, this is preferably done through universal amplification of the various nucleic acid regions to be analyzed using the assay systems of the invention. Universal primer sequences are added to the selectively amplified nucleic acid regions so that they may be further amplified in a single universal amplification reaction. These universal primer sequences may be added to the nucleic acids regions during the selective amplification process, i.e., the primers for selective amplification have universal primer sequences that flank a locus. Alternatively, adapters comprising universal amplification sequences can be added to the ends of the selected nucleic acids as adapters following amplification and isolation of the selected nucleic acids from the mixed sample.

In one exemplary aspect, nucleic acids are initially amplified or isolated from a maternal sample using primers complementary to selected regions of the sex chromosomes, followed by a universal amplification step to increase the number of nucleic acid regions for analysis. In a preferred aspect the universal amplification step is universal PCR. This introduction of primer regions to the initial amplification products from a maternal sample allows a subsequent controlled universal amplification of all or a portion of selected nucleic acids prior to or during analysis, e.g., sequence determination.

Bias and variability can be introduced during DNA amplification, such as that seen during polymerase chain reaction (PCR). In cases where an amplification reaction is multiplexed, there is the potential that loci will amplify at different rates or efficiency. Part of this may be due to the variety of primers in a multiplex reaction with some having better efficiency (i.e. hybridization) than others, or some working better in specific experimental conditions due to the base composition. Each set of primers for a given locus may behave differently based on sequence context of the primer and template DNA, buffer conditions, and other conditions. A universal DNA amplification for a multiplexed assay system will generally introduce less bias and variability.

Accordingly, in a preferred aspect, a small number (e.g., 1-10, preferably 3-5) of cycles of selected amplification or nucleic acid enrichment in a multiplexed mixture reaction are performed, followed by universal amplification using introduced universal primers. The number of cycles using universal primers will vary, but will preferably be at least 10 cycles, more preferably at least 5 cycles, even more preferably 20 cycles or more. By moving to universal amplification following a lower number of amplification cycles, the bias of having certain loci amplify at greater rates than others is reduced.

Optionally, the assay system will include a step between the selected isolation and/or amplification and universal amplification to remove any excess nucleic acids that are not specifically amplified in the selected amplification.

The whole product or an aliquot of the product from the selected amplification may be used for the universal amplification. The same or different conditions (e.g., polymerase, buffers, and the like) may be used in the amplification steps, e.g., to ensure that bias and variability is not inadvertently introduced due to experimental conditions. In addition, variations in primer concentrations may be used to effectively limit the number of sequence specific amplification cycles.

In certain aspects, the universal primer regions of the primers or adapters used in the assay system are designed to be compatible with conventional multiplexed assay methods that utilize general priming mechanisms to analyze large numbers of nucleic acids simultaneously in one reaction in one vessel. Such “universal” priming methods allow for efficient, high volume analysis of the quantity of nucleic acid regions present in a mixed sample, and allow for comprehensive quantification of the presence of nucleic acid regions within such a mixed sample for the determination of aneuploidy.

Examples of such assay methods include, but are not limited to, multiplexing methods used to amplify and/or genotype a variety of samples simultaneously, such as those described in Oliphant et al., U.S. Pat. No. 7,582,420 and Oliphant et al., U.S. Ser. Nos. 13/013,732, 13/205,570 and 13/205,603, which are incorporated by reference herein.

Some aspects utilize coupled reactions for multiplex detection of nucleic acid sequences where oligonucleotides from an early phase of each process contain sequences which may be used by oligonucleotides from a later phase of the process. Exemplary processes for amplifying and/or detecting nucleic acids in samples can be used, alone or in combination, including but not limited to the methods described below, each of which are incorporated by reference in their entirety.

In certain aspects, the assay system of the invention utilizes one of the following combined selective and universal amplification techniques: (1) LDR coupled to PCR; (2) primary PCR coupled to secondary PCR coupled to LDR; and (3) primary PCR coupled to secondary PCR. Each of these aspects of the invention has particular applicability in detecting certain nucleic acid characteristics. However, each requires the use of coupled reactions for multiplex detection of nucleic acid sequence differences where oligonucleotides from an early phase of each process contain sequences which may be used by oligonucleotides from a later phase of the process.

Barany et al., U.S. Pat. Nos. 6,852,487, 6,797,470, 6,576,453, 6,534,293, 6,506,594, 6,312,892, 6,268,148, 6,054,564, 6,027,889, 5,830,711, 5,494,810, describe the use of the ligase chain reaction (LCR) assay for the detection of specific sequences of nucleotides in a variety of nucleic acid samples.

Barany et al., U.S. Pat. Nos. 7,807,431, 7,455,965, 7,429,453, 7,364,858, 7,358,048, 7,332,285, 7,320,865, 7,312,039, 7,244,831, 7,198,894, 7,166,434, 7,097,980, 7,083,917, 7,014,994, 6,949,370, 6,852,487, 6,797,470, 6,576,453, 6,534,293, 6,506,594, 6,312,892, and 6,268,148 describe the use of the ligase detection reaction with detection reaction (“LDR”) coupled with polymerase chain reaction (“PCR”) for nucleic acid detection.

Barany et al., U.S. Pat. Nos. 7,556,924 and 6,858,412, describe the use of padlock probes (also called “precircle probes” or “multi-inversion probes”) with coupled ligase detection reaction (“LDR”) and polymerase chain reaction (“PCR”) for nucleic acid detection.

Barany et al., U.S. Pat. Nos. 7,807,431, 7,709,201, and 7,198,814 describe the use of combined endonuclease cleavage and ligation reactions for the detection of nucleic acid sequences.

Willis et al., U.S. Pat. Nos. 7,700,323 and 6,858,412, describe the use of precircle probes in multiplexed nucleic acid amplification, detection and genotyping, including

Ronaghi et al., U.S. Pat. No. 7,622,281 describes amplification techniques for labeling and amplifying a nucleic acid using an adapter comprising a unique primer and a barcode.

In addition to the various amplification techniques, numerous methods of sequence determination are compatible with the assay systems of the inventions. Preferably, such methods include “next generation” methods of sequencing. Exemplary methods for sequence determination include, but are not limited to, including, but not limited to, hybridization-based methods, such as disclosed in Drmanac, U.S. Pat. Nos. 6,864,052; 6,309,824; and 6,401,267; and Drmanac et al, U.S. patent publication 2005/0191656, which are incorporated by reference, sequencing by synthesis methods, e.g., Nyren et al, U.S. Pat. Nos. 7,648,824, 7,459,311 and 6,210,891; Balasubramanian, U.S. Pat. Nos. 7,232,656 and 6,833,246; Quake, U.S. Pat. No. 6,911,345; Li et al, Proc. Natl. Acad. Sci., 100: 414-419 (2003); pyrophosphate sequencing as described in Ronaghi et al., U.S. Pat. Nos. 7,648,824, 7,459,311, 6,828,100, and 6,210,891; and ligation-based sequencing determination methods, e.g., Drmanac et al., U.S. Pat. Appln No. 20100105052, and Church et al, U.S. Pat. Appln Nos. 20070207482 and 20090018024.

Alternatively, nucleic acid regions of interest can be selected and/or identified using hybridization techniques. Methods for conducting polynucleotide hybridization assays for detection of have been well developed in the art. Hybridization assay procedures and conditions will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred aspects. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

### Use of Indices in the Assay Systems of the Invention

In certain aspects, all or a portion of the sequences of the nucleic acids of interest are directly detected using the described techniques, e.g., sequence determination or hybridization. In certain aspects, however, the nucleic acids of interest are associated with one or more indices that are identifying for a selected nucleic acid region or a particular sample being analyzed. The detection of the one or more indices can serve as a surrogate detection mechanism of the selected nucleic acid region, or as confirmation of the presence of a particular selected nucleic acid region if both the sequence of the index and the sequence of the nucleic acid region itself are determined. These indices are preferably associated with the selected nucleic acids during an amplification step using primers that comprise both the index and sequence regions that specifically hybridize to the nucleic acid region.

In one example, the primers used for amplification of a selected nucleic acid region are designed to provide a locus index between the selected nucleic acid region primer region and a universal amplification region. The locus index is unique for each selected nucleic acid region and representative of a locus on a sex chromosome or reference chromosome, so that quantification of the locus index in a sample provides quantification data for the locus and the particular chromosome containing the locus.

In another example, the primers used for amplification of a selected nucleic acid region are designed to provide an allele index between the selected nucleic acid region primer region and a universal amplification region. The allele index is unique for particular alleles of a selected nucleic acid region and representative of a locus variation present on a sex chromosome or reference chromosome, so that quantification of the allele index in a sample provides quantification data for the allele and the summation of the allelic indices for a particular locus provides quantification data for both the locus and the particular chromosome containing the locus.

In another aspect, the primers used for amplification of the selected nucleic acid regions to be analyzed for a mixed sample are designed to provide an identification index between the selected nucleic acid region primer region and a universal amplification region. In such an aspect, a sufficient number of identification indices are present to uniquely identify each selected nucleic acid region in the sample. Each nucleic acid region to be analyzed is associated with a unique identification index, so that the identification index is uniquely associated with the selected nucleic acid region. Quantification of the identification index in a sample provides quantification data for the associated selected nucleic acid region and the chromosome corresponding to the selected nucleic acid region. The identification locus may also be used to detect any amplification bias that occurs downstream of the initial isolation of the selected nucleic acid regions from a sample.

In certain aspects, only the locus index and/or the identification index (if present) are detected and used to quantify the selected nucleic acid regions in a sample. In another aspect, a count of the number of times each locus index occurs with a unique identification index is done to determine the relative frequency of a selected nucleic acid region in a sample.

In some aspects, indices representative of the sample from which a nucleic acid is isolated are used to identify the source of the nucleic acid in a multiplexed assay system. In such aspects, the nucleic acids are uniquely identified with the sample index. Those uniquely identified oligonucleotides may then be combined into a single reaction vessel with nucleic acids from other samples prior to sequencing. The sequencing data is first segregated by each unique sample index prior to determining the frequency of each target locus for each sample and prior to determining whether there is a chromosomal abnormality for each sample. For detection, the sample indices, the locus indices, and the identification indices (if present), are sequenced.

In aspects of the invention using indices, the selective amplification primers are preferably designed so that indices comprising identifying information are coded at one or both ends of the primer. Alternatively, the indices and universal amplification sequences can be added to the selectively amplified nucleic acids following initial amplification.

The indices are non-complementary but unique sequences used within the primer to provide information relevant to the selective nucleic acid region that is isolated and/or amplified using the primer. The advantage of this is that information on the presence and quantity of the selected nucleic acid region can be obtained without the need to determine the actual sequence itself, although in certain aspects it may be desirable to do so. Generally, however, the ability to identify and quantify a selected nucleic acid region through identification of one or more indices will decrease the length of sequencing required as the loci information is captured at the 3′ or 5′ end of the isolated selected nucleic acid region. Use of indices identification as a surrogate for identification of selected nucleic acid regions may also reduce error since longer sequencing reads are more prone to the introduction or error.

In addition to locus indices, allele indices and identification indices, additional indices can be introduced to primers to assist in the multiplexing of samples. For example, correction indices which identify experimental error (e.g., errors introduced during amplification or sequence determination) can be used to identify potential discrepancies in experimental procedures and/or detection methods in the assay systems. The order and placement of these indices, as well as the length of these indices, can vary, and they can be used in various combinations.

The primers used for identification and quantification of a selected nucleic acid region may be associated with regions complementary to the 5′ of the selected nucleic acid region, or in certain amplification regimes the indices may be present on one or both of a set of amplification primers which comprise sequences complementary to the sequences of the selected nucleic acid region. The primers can be used to multiplex the analysis of multiple selected nucleic acid regions to be analyzed within a sample, and can be used either in solution or on a solid substrate, e.g., on a microarray or on a bead. These primers may be used for linear replication or amplification, or they may create circular constructs for further analysis.

### Variation Minimization Within and Between Samples

One challenge with the detection of chromosomal abnormalities in a mixed sample is that often the DNA from the cell type with the putative chromosomal abnormality is present in much lower abundance than the DNA from normal cell type. In the case of a mixed maternal sample containing fetal and maternal cell free DNA, the cell free fetal DNA as a percentage of the total cell free DNA may vary from less than one to forty percent, and most commonly is present at or below twenty percent and frequently at or below ten percent. In the detection of an aneuploidy such as Trisomy X in the fetal DNA of such mixed maternal sample, the relative increase in Chromosome X is 50% in the fetal DNA and thus as a percentage of the total DNA in a mixed sample where, as an example, the fetal DNA is 5% of the total, the increase in Chromosome X as a percentage of the total is 2.5%. If one is to detect this difference robustly through the methods described herein, the variation in the measurement of Chromosome X has to be much less than the percent increase of Chromosome X.

The variation between levels found between samples and/or for nucleic acid regions within a sample may be minimized in a combination of analytical methods, many of which are described in this application. For instance, variation is lessened by using an internal reference in the assay. An example of an internal reference is the use of a chromosome present in a “normal” abundance (e.g., disomy for an autosome) to compare against a chromosome present in putatively abnormal abundance, such as aneuploidy, in the same sample. While the use of one such “normal” chromosome as a reference chromosome may be sufficient, it is also possible to use many normal chromosomes as the internal reference chromosomes to increase the statistical power of the quantification.

One method of using an internal reference is to calculate a ratio of abundance of the putatively abnormal chromosomes to the abundance of the normal chromosomes in a sample, called a chromosomal ratio. In calculating the chromosomal ratio, the abundance or counts of each of the nucleic acid regions for each chromosome are summed together to calculate the total counts for each chromosome. The total counts for one chromosome are then divided by the total counts for a different chromosome to create a chromosomal ratio for those two chromosomes.

Alternatively, a chromosomal ratio for each chromosome may be calculated by first summing the counts of each of the nucleic acid regions for each chromosome, and then dividing the sum for one chromosome by the total sum for two or more chromosomes. Once calculated, the chromosomal ratio is then compared to the average chromosomal ratio from a normal population.

The average may be the mean, median, mode or other average, with or without normalization and exclusion of outlier data. In a preferred aspect, the mean is used. In developing the data set for the chromosomal ratio from the normal population, the normal variation of the measured chromosomes is calculated. This variation may be expressed a number of ways, most typically as the coefficient of variation, or CV. When the chromosomal ratio from the sample is compared to the average chromosomal ratio from a normal population, if the chromosomal ratio for the sample falls statistically outside of the average chromosomal ratio for the normal population, the sample contains an aneuploidy. The criteria for setting the statistical threshold to declare an aneuploidy depend upon the variation in the measurement of the chromosomal ratio and the acceptable false positive and false negative rates for the desired assay. In general, this threshold may be a multiple of the variation observed in the chromosomal ratio. In one example, this threshold is three or more times the variation of the chromosomal ratio. In another example, it is four or more times the variation of the chromosomal ratio. In another example it is five or more times the variation of the chromosomal ratio. In another example it is six or more times the variation of the chromosomal ratio. In the example above, the chromosomal ratio is determined by summing the counts of nucleic acid regions by chromosome. Typically, the same number of nucleic acid regions for each chromosome is used. An alternative method for generating the chromosomal ratio would be to calculate the average counts for the nucleic acid regions for each chromosome. The average may be any estimate of the mean, median or mode, although typically an average is used. The average may be the mean of all counts or some variation such as a trimmed or weighted average. Once the average counts for each chromosome have been calculated, the average counts for each chromosome may be divided by the other to obtain a chromosomal ratio between two chromosomes, the average counts for each chromosome may be divided by the sum of the averages for all measured chromosomes to obtain a chromosomal ratio for each chromosome as described above. As highlighted above, the ability to detect an aneuploidy in a mixed sample where the putative DNA is in low relative abundance depends greatly on the variation in the measurements of different nucleic acid regions in the assay. Numerous analytical methods can be used which reduce this variation and thus improve the sensitivity of this method to detect aneuploidy. One method for reducing variability of the assay is to increase the number of nucleic acid regions used to calculate the abundance of the chromosomes. In general, if the measured variation of a single nucleic acid region of a chromosome is X % and Y different nucleic acid regions are measured on the same chromosome, the variation of the measurement of the chromosomal abundance calculated by summing or averaging the abundance of each nucleic acid region on that chromosome will be approximately X % divided by Ŷ1/2. Stated differently, the variation of the measurement of the chromosome abundance would be approximately the average variation of the measurement of each nucleic acid region's abundance divided by the square root of the number of nucleic acid regions.

In a preferred aspect of this invention, the number of nucleic acid regions measured for each chromosome (and in the sex chromosomes, for the PARs) is at least 24. In another preferred aspect of this invention, the number of nucleic acid regions measured for each chromosome is at least 48. In another preferred aspect of this invention, the number of nucleic acid regions measured for each chromosome is at least 100. In another preferred aspect of this invention the number of nucleic acid regions measured for each chromosome is at least 200. There is incremental cost to measuring each nucleic acid region and thus it is important to minimize the number of each nucleic acid region. In a preferred aspect of this invention, the number of nucleic acid regions measured for each chromosome is less than 2000. In a preferred aspect of this invention, the number of nucleic acid regions measured for each chromosome is less than 1000. In a most preferred aspect of this invention, the number of nucleic acid regions measured for each chromosome is at least 48 and less than 1000. In one aspect, following the measurement of abundance for each nucleic acid region, a subset of the nucleic acid regions may be used to determine the presence or absence of aneuploidy. There are many standard methods for choosing the subset of nucleic acid regions. These methods include outlier exclusion, where the nucleic acid regions with detected levels below and/or above a certain percentile are discarded from the analysis. In one aspect, the percentile may be the lowest and highest 5% as measured by abundance. In another aspect, the percentile may be the lowest and highest 10% as measured by abundance. In another aspect, the percentile may be the lowest and highest 25% as measured by abundance.

Another method for choosing the subset of nucleic acid regions include the elimination of regions that fall outside of some statistical limit. For instance, regions that fall outside of one or more standard deviations of the mean abundance may be removed from the analysis. Another method for choosing the subset of nucleic acid regions may be to compare the relative abundance of a nucleic acid region to the expected abundance of the same nucleic acid region in a healthy population and discard any nucleic acid regions that fail the expectation test. To further minimize the variation in the assay, the number of times each nucleic acid region is measured may be increased. As discussed, in contrast to the random methods of detecting aneuploidy where the genome is measured on average less than once, the assay systems of the present invention intentionally measures each nucleic acid region multiple times. In general, when counting events, the variation in the counting is determined by Poisson statistics, and the counting variation is typically equal to one divided by the square root of the number of counts. In a preferred aspect of the invention, the nucleic acid regions are each measured on average at least 100 times. In a preferred aspect to the invention, the nucleic acid regions are each measured on average at least 500 times. In a preferred aspect to the invention, the nucleic acid regions are each measured on average at least 1000 times. In a preferred aspect to the invention, the nucleic acid regions are each measured on average at least 2000 times. In a preferred aspect to the invention, the nucleic acid regions are each measured on average at least 5000 times.

In another aspect, subsets of loci can be chosen randomly but with sufficient numbers of loci to yield a statistically significant result in determining the sex of the fetus or whether a sex chromosomal abnormality exists. Multiple analyses of different subsets of loci can be performed within a mixed sample to yield more statistical power. In this example, it may or may not be necessary to remove or eliminate any loci prior to the random analysis. For example, if there are 100 selected regions for chromosome 21 and 100 selected regions for chromosome 18, a series of analyses could be performed that evaluate fewer than 100 regions for each of the chromosomes.

In addition to the methods above for reducing variation in the assay, other analytical techniques, many of which are described earlier in this application, may be used in combination. In general, the variation in the assay may be reduced when all of the nucleic acid regions for each sample are interrogated in a single reaction in a single vessel. Similarly, the variation in the assay may be reduced when a universal amplification system is used. Furthermore, the variation of the assay may be reduced when the number of cycles of amplification is limited.

### Computer Implementation of the Processes of the Invention

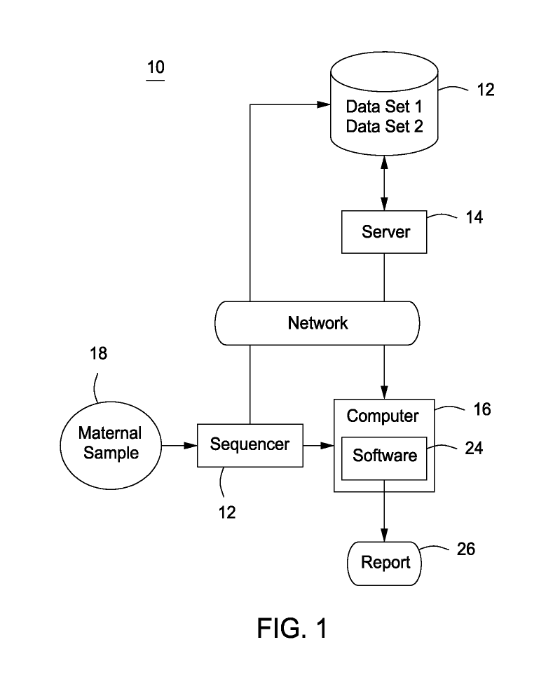

FIG. 1 is a block diagram illustrating an exemplary system environment in which the processes of the present invention may be implemented. The system 10 includes a server 14 and a computer 16, and preferably these are associated with a DNA sequencer 12. The DNA sequencer 12 may be coupled to the server 14 and/or the computer directly or through a network. The computer 16 may be in communication with the server 14 through the same or different network.

The DNA sequencer 12 may be any commercially available instrument that automates the DNA sequencing process for sequence analysis of nucleic acids representative of a nucleic acid in the maternal sample 18. The output of the DNA sequencer 12 may be in the form of multiplexed data sets 20 comprising frequency data for loci and/or samples, and optionally these are distinguishable based on associated indices. In one embodiment, the multiplexed data set 20 may be stored in a database 22 that is accessible by the server 14.

According to the exemplary embodiment, the computer 16 executes a software component 24 that calculates the relative frequencies of the genomic regions and/or chromosomes from a maternal sample 18. In one embodiment, the computer 16 may comprise a personal computer, but the computer 16 may comprise any type of machine that includes at least one processor and memory.

The output of the software component 24 comprises a report 26 with a relative frequency of a genomic region and/or a chromosome and/or results of the comparison of such genomic regions and/or chromosomes. The report 26 may be paper that is printed out, or electronic, which may be displayed on a monitor and/or communicated electronically to users via e-mail, FTP, text messaging, posted on a server, and the like.

Although the processes of the invention are shown as being implemented as software 24, they can also be implemented as a combination of hardware and software. In addition, the software 24 may be implemented as multiple components operating on the same or different computers.

Both the server 14 and the computer 16 may include hardware components of typical computing devices (not shown), including a processor, input devices (e.g., keyboard, pointing device, microphone for voice commands, buttons, touchscreen, etc.), and output devices (e.g., a display device, speakers, and the like). The server 14 and computer 16 may include computer-readable media, e.g., memory and storage devices (e.g., flash memory, hard drive, optical disk drive, magnetic disk drive, and the like) containing computer instructions that implement the functionality disclosed when executed by the processor. The server 14 and the computer 16 may further include wired or wireless network communication interfaces for communication.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example 1

### Subjects

Subjects are prospectively enrolled upon providing informed consent under protocols approved by institutional review boards. Subjects are required to be at least 18 years of age, at least 10 weeks gestational age, and to have singleton pregnancies.

### Example 2

### Analysis of Polymorphic Loci to Assess Percent Fetal Contribution

To assess fetal nucleic acid proportion in the maternal samples, assays are designed against a set of 192 SNP-containing loci on chromosomes 1 through 12, where two middle oligos differing by one base are used to query each SNP. SNPs are optimized for minor allele frequency in the HapMap 3 dataset. Duan, et al., Bioinformation, 3(3):139-41 (2008); Epub 2008 Nov. 9.

Oligonucleotides are synthesized by IDT and pooled together to create a single multiplexed assay pool. PCR products are generated from each subject sample as previously described. Briefly, 8 mL blood per subject is collected into a Cell-free DNA tube (Streck) and stored at room temperature for up to 3 days. Plasma is isolated from blood via double centrifugation and stored at −20C for up to a year. cfDNA is isolated from plasma using Viral NA DNA purification beads (Dynal), biotinylated, immobilized on MyOne™ C1 streptavidin beads (Life Technologies, Carlsbad, Calif.), and annealed with the multiplexed oligonucleotide pool. Appropriately hybridized oligonucleotides are catenated with Taq ligase, eluted from the cfDNA, and amplified using universal PCR primers. PCR product from 96 independent samples is pooled and used as template for cluster amplification on a single lane of a TruSeq™ v3 SR flow slide (Illumina, San Diego, Calif.). The slide is processed on an Illumina HiSeq™ 2000 to produce a 56 base locus-specific sequence and a 7 base sample tag sequence from an average of 1.18M clusters/sample. Locus-specific reads are compared to expected locus sequences.

Informative polymorphic loci are defined as loci where fetal alleles differed from maternal alleles. Because the assay exhibits allele specificities exceeding 99%, informative loci are readily identified when the fetal allele proportion of a locus is measured to be between 1 and 20%. A maximum likelihood is estimated using a binomial distribution, such as that described in co-pending application 61/509,188, to determine the most likely fetal proportion based upon measurements from several informative loci. The results correlated well (R2>0.99) with the weighted average approach presented by Chu and colleagues (see, Chu, et al., Prenat. Diagn., 30:1226-29 (2010)).

### Example 3

### Analysis of PARs to Determine Aneuploidy

Because the sequences from the PAR region are found in both the X and Y chromosome, the dosage of the PAR regions will reflect a disomic level of sex chromosomes in both a normal male and normal female fetus. The level of sex chromosomes can be determined by using a reference chromosome and comparison of the genetic dosage of the PAR regions as compared to the dosage of a disomic reference chromosome.

The levels estimated are thus levels of the overall number of sex chromosomes, but do not distinguish between a Y chromosome and an X chromosome.

To estimate fetal chromosome dosage of the sex chromosome and a reference chromosome (e.g., any individual chromosome other than X), assays are designed against 576 non-polymorphic loci within the pseudoautosomal region and 576 non-polymorphic loci on one or more reference chromosomes. Each assay utilizes three locus-specific oligonucleotides: a left oligo with a 5′ universal amplification tail, a 5′ phosphorylated middle oligo, and a 5′ phosphorylated right oligo with a 3′ universal amplification tail. The selected loci are used to compute a fetal contribution dosage metric for sex chromosomes by utilizing the PAR dosage. Sequence counts are normalized by systematically removing sample and assay biases using median polish (see Tukey, Exploratory Data Analysis (Addison-Wesley, Reading Mass., 1977) and Irzarry, et al., NAR, 31(4):e15 (2003)).

### Example 4

### Assessment of Fetal Chromosome Contribution of Fetal Sex Chromosomes

Assays are designed against a set of 20 SNP-containing loci on chromosome X outside the PAR region, 20 SNP-containing loci on chromosome X within the PAR region, and 20 SNP containing loci on a comparator chromosome (e.g., chromosome 2). A comparison of determined levels within a PAR in a maternal sample to the determined levels of chromosome 2 is used to assess the total fetal contribution of the sex chromosomes in the maternal sample. A comparison of the contribution of the sex chromosomes, as determined by the detected loci within the PAR, and the determined levels from loci on the X chromosome outside the PAR is used to calculate the contribution from a fetal X versus from a fetal Y. The assay is thus used to simultaneously identify the presence or absence of an aneuploidy in the sex chromosomes, the nature of such aneuploidy, and the sex of the fetus.

Each assay consists of three locus specific oligonucleotides: a left oligo with a 5′ universal amplification tail, a 5′ phosphorylated middle oligo, and a 5′ phosphorylated right oligo with a 3′ universal amplification tail. Two middle oligos differing by one base are used to query each SNP in the selected loci. SNPs are optimized for minor allele frequency in the HapMap 3 dataset. Duan, et al., Bioinformation, 3(3):139-41 (2008); Epub 2008 Nov. 9.

Oligonucleotides are synthesized by IDT (Coralville, Iowa) and pooled together to create a single multiplexed assay pool. PCR products are generated from each subject sample as described in U.S. Ser. Nos., 13/013,732, 13/205,490, 13/205,570, and 13/205,603, filed Aug. 8, 2011, each of which are incorporated by reference in their entirety. Briefly, 8 ml blood per subject is collected into a glass tube comprising preservatives (Streck, Omaha, Nebr.) and stored at room temperature for up to 3 days. Plasma is isolated from blood via double centrifugation and stored at −20° C. for up to a year. cfDNA is isolated from plasma using Viral NA DNA purification beads (Life Technologies, Carlsbad, Calif.), biotinylated, immobilized on MyOne C1 streptavidin beads (Life Technologies, Carlsbad, Calif.), and annealed with the multiplexed oligonucleotide pool. Appropriately hybridized oligonucleotides are catenated with Taq ligase, eluted from the cfDNA, and amplified using universal PCR primers. PCR products from 96 independent samples are pooled and used as template for cluster amplification on a single lane of a TruSeq™ V3 SR flow slide (Illumina, San Diego, Calif.). The slide is processed on an Illumina HiSeq™ 2000 to produce a 56 base locus-specific sequence and a 7 base sample tag sequence from an average of 1.18M clusters/sample.

A maximum likelihood is estimated using a binomial distribution, such as that described in co-pending application 61/509,188, to determine the most likely fetal dosage of chromosome X and collective fetal dosage of chromosome 2 based upon measurements from the informative loci. Since chromosome 2 is not expected to exhibit any evidence of aneuploidy, the comparator of overall levels of chromosome X (as determined by the PAR loci) is used for determining the risk of either monosomy or trisomy of the sex chromosomes. A further comparison with the X loci outside the PAR is used to determine the percentage of PAR loci from the X chromosome versus the Y chromosome. Samples from a normal male are distinguished from a sample from a Turner's Syndrome female, as the ratio of loci from the PAR region and the X chromosome outside the PAR region is 1:1 in a normal male and 1:0.5 in a Turner syndrome female. In addition, the comparison of the PAR and non-PAR X loci allows the identification of particular trisomies, as the ratio can distinguish between a triple X, an XXY, or an XYY.

### Example 5

### Sex Determination

The frequency of loci from different regions of the X chromosome can be used directly in sex determination. Assays are designed against a set of 20 SNP-containing loci on chromosome X outside the PAR region and 20 SNP-containing loci on chromosome X within the PAR region. The frequency of these regions can be determined as described in Example 4.

A comparison of determined levels within a PAR region in a maternal sample to the determined levels of loci on chromosome X outside the PAR region is used to assess the sex of the fetus. Specifically, the comparison of the contribution of the sex chromosomes, as determined by the detected loci within the PAR, and the determined levels from loci on the X chromosome outside the PAR, can differentiate between an XX genotype and an XY genotype, as the ratio of the PAR to non-PAR loci should effectively be 1:1 in XX fetus and 1:0.05 in an XY fetus when the percent fetal is 10%.

The presence of an XO phenotype is optionally determined as well to rule out the possibility that the difference is ratio is due to monosomy X rather than the XY genotype. The assay system of the invention can thus be used to simultaneously identify the presence or absence of an aneuploidy in the sex chromosomes and the sex of the fetus.

### Example 6

### Identification of Monosomy X

In some aspects, the assay system is used to identify a monosomy X genotype in a fetus. The mean of counts from the 384 loci are divided by the sum of the mean counts for the 384 chromosome X loci and mean counts for all 576 loci from the reference chromosome. A reference chromosome proportion metric is calculated using all 576 loci from the reference chromosome.

A standard Z test of proportions is used to compute Z statistics:

$${Z}_{j} = \frac{{p}_{j} - {p}_{0}}{\sqrt{\frac{{p}_{j} ( 1 - {p}_{j} )}{{n}_{j}}}}$$

where pⱼ is the observed proportion for the X chromosome in a given sample j, p₀ is the expected proportion for the X chromosome calculated as the median pⱼ, and nⱼ is the denominator of the proportion metric. Z statistic standardization is performed using iterative censoring. At each iteration, the samples falling outside of three median absolute deviations are removed. After ten iterations, mean and standard deviation are calculated using only the uncensored samples. All samples are then standardized against this mean and standard deviation. The Kolmogorov-Smirnov test (see Conover, Practical Nonparametric Statistics, pp. 295-301 (John Wiley & Sons, New York, N.Y., 1971)) and Shapiro-Wilk's test (see Royston, Applied Statistics, 31:115-124 (1982)) are used to test for the normality of the normal samples' Z statistics.

### Example 7

### Identification of Sex Chromosome Trisomy

The 384 loci within the PAR from normal XX or XY samples and trisomic sex chromosome samples are identified using Z Statistics derived from individual loci. The mean of counts from the 384 loci are divided by the sum of the mean count for the 384 PAR loci and mean count for all 576 loci from the reference chromosome. A reference chromosome proportion metric is calculated using all 576 loci from the reference chromosome.

A standard Z test of proportions is used to compute Z statistics:

$${Z}_{j} = \frac{{p}_{j} - {p}_{0}}{\sqrt{\frac{{p}_{j} ( 1 - {p}_{j} )}{{n}_{j}}}}$$

where pⱼ is the observed proportion for the sex chromosomes in a given sample j, p₀ is the expected proportion for the sex chromosome calculated as the median pⱼ, and nⱼ is the denominator of the proportion metric. Z statistic standardization is performed using iterative censoring. At each iteration, the samples falling outside of three median absolute deviations are removed. After ten iterations, mean and standard deviation are calculated using only the uncensored samples. All samples are then standardized against this mean and standard deviation. The Kolmogorov-Smirnov test (see Conover, Practical Nonparametric Statistics, pp. 295-301 (John Wiley & Sons, New York, N.Y., 1971)) and Shapiro-Wilk's test (see Royston, Applied Statistics, 31:115-124 (1982)) are used to test for the normality of the normal samples' Z statistics.

### Example 8

### Aneuploidy Detection Using Risk Calculation

The risk of aneuploidy is calculated using an odds ratio that compares a model assuming a disomic fetal chromosome and a model assuming either a monosomic or trisomic fetal sex chromosome. The distribution of differences in observed and reference proportions are evaluated using normal distributions with a mean of 0 and standard deviation estimated using Monte Carlo simulations that randomly draw from observed data. For the disomic model, p₀ is used as the expected reference proportion in the simulations. For the monosomic or trisomic models, p₀ is adjusted on a per sample basis with the fetal proportion adjusted reference proportion \hat{p}ⱼ, defined as

$${\hat{p}}_{j} = \frac{( 1 + 0.5 {f}_{j} ) {p}_{0}}{( ( 1 + 0.5 {f}_{j} ) {p}_{0} ) + ( 1 - {p}_{0} )}$$

where fⱼ is the fetal proportion for sample j. This adjustment accounts for the expected changes in representation of a test chromosome when the fetus has an aneuploidy. In the simulations both p₀ and fⱼ are randomly chosen from normal distributions using their mean and standard error estimates to account for measurement variances. Simulations are executed 100,000 times. The risk score is defined as the mean aneuploidy versus disomy odds ratio obtained from the simulations, adjusted by multiplying the risk of aneuploidy associated with the subject's maternal and gestational age.

### Example 9

### Aneuploidy Detection Using Risk Calculation

The risk calculation algorithm used in calculation of the estimated risk of aneuploidy uses an odds ratio comparing a mathematic model assuming a disomic fetal chromosome and a mathematic model assuming a monosomic or trisomic fetal chromosome. When x=pⱼ−p₀, is used to describe the difference of the observed proportion pⱼ for sample j and the estimated reference proportion p₀, the risk calculation algorithm computes:

$$\frac{P ( {x}_{j} \vert A )}{P ( {x}_{j} \vert D )} ,$$

where A is the aneuploid model and D is the disomic model. The disomic model D is a normal distribution with mean 0 and a sample specific standard deviation estimated by Monte Carlo simulations as described below. The aneuploid model A is also a normal distribution with mean 0, determined by transforming xⱼ to \hat{x}ⱼ=pⱼ−\hat{p}ⱼ, the difference between the observed proportion and a fetal fraction adjusted reference proportion as defined by:

$${\hat{p}}_{j} = \frac{( 1 + 0.5 {f}_{j} ) {p}_{0}}{( 1 + 0.5 {f}_{j} ) {p}_{o} + ( 1 - {p}_{0} )} .$$

where fⱼ is the fetal fraction for sample j. This adjustment accounted for the expected increased representation of an aneuploidy fetal sex chromosome. Monte Carlo simulations are used to estimate sample specific standard deviations for disomic and aneuploid models of proportion differences. Observed proportions for each sample are simulated by non-parametric bootstrap sampling of loci and calculating means, or parametric sampling from a normal distribution using the mean and standard error estimates for each chromosome from the observed non-polymorphic locus counts. Similarly, the reference proportion p₀ and fetal fraction fⱼ, are simulated by non-parametric sampling of samples and polymorphic loci respectively, or chosen from normal distributions using their mean and standard error estimates to account for measurement variances. Parametric sampling is used in this study. Simulations are executed 100,000 times, and proportion differences are computed for each execution to construct the distributions. Based on the results of these simulations, normal distributions are found to be good models of disomy and trisomy.

The final risk calculation algorithm risk score is defined as

$$\frac{P ( {x}_{j} \vert A ) P ( A )}{P ( {x}_{j} \vert D ) P ( D )}$$

where P(A)/P(D) is the prior risk of aneuploidy vs. disomy. The data on prior risk of aneuploidy is taken from well-established tables capturing the risk associated with the subject's maternal and gestational age (Nicolaides K H. Screening for chromosomal defects. Ultrasound Obstet Gynecol 2003; 21:313-321).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. §112, ¶6.

What is claimed is:

1. An assay system for detection of the presence or absence of a sex chromosome aneuploidy comprising the steps of:
providing a biological sample containing DNA;
amplifying one or more selected nucleic acid regions from a pseudoautosomal region in the biological sample;
amplifying one or more selected nucleic acid regions from an autosomal region in the biological sample;
detecting the amplified nucleic acid regions;
quantifying the relative frequency of the selected nucleic acid regions from the pseudoautosomal and autosomal regions;
comparing the relative frequency of the selected nucleic acid regions from the pseudoautosomal and autosomal regions; and
identifying the presence or absence of an aneuploidy of a sex chromosome based on the compared relative frequencies of the pseudoautosomal and autosomal regions.

2. An assay system for detection of the presence or absence of a sex chromosome aneuploidy comprising the steps of:
providing a mixed sample comprising cell free DNA;
amplifying two or more selected nucleic acid regions from a pseudoautosomal region in the mixed sample;
amplifying two or more selected nucleic acid regions from an autosomal region in the mixed sample;
detecting the amplified nucleic acid regions;
quantifying the relative frequency of the selected nucleic acid regions from the pseudoautosomal and autosomal regions;
comparing the relative frequency of the selected nucleic acid regions from the pseudoautosomal and autosomal regions; and
identifying the presence or absence of an aneuploidy of a sex chromosome based on the compared relative frequencies of the pseudoautosomal and autosomal regions.

3. The assay system of claim 2, wherein the relative frequencies of the selected nucleic acid regions are individually quantified, and the relative frequencies of the individual nucleic acid regions are compared to determine the presence or absence of a sex chromosome aneuploidy.

4. The assay system of claim 2, wherein the comparison of the relative frequencies of the pseudoautosomal and autosomal regions is expressed as a chromosomal ratio.

5. The assay system of claim 4, wherein the chromosomal ratio is compared to the mean chromosomal ratio from a reference population and the threshold for identifying the presence or absence of an aneuploidy is at least three times the chromosomal variation of the reference population.

6. The assay system of claim 2, wherein the quantified relative frequencies of the nucleic acid regions are used to determine a chromosome frequency of one or both of the sex chromosomes, and wherein the presence or absence of an aneuploidy is based on the compared chromosome frequencies.

7. The assay system of claim 2, wherein the quantified relative frequencies of the selected nucleic acid regions are normalized following detection and prior to quantification.

8. The assay system of claim 7, wherein the relative frequencies of each nucleic acid region for each chromosome are summed and the sums for each chromosome are compared to calculate a chromosomal ratio.

9. The assay system of claim 8, wherein the chromosomal ratio is compared to the mean chromosomal ratio from a normal population and the threshold for identifying the presence or absence of an aneuploidy is at least three times the chromosomal variation in a normal population.

10. The assay system of claim 2, where the nucleic acid regions are assayed in a single vessel.

11. The assay system of claim 2, where the nucleic acid regions undergo a universal amplification.

12. The assay system of claim 2, where the nucleic acid regions are each counted an average of at least 500 times.

13. The assay system of claim 2, wherein the frequency of non-pseudoautosomal regions of the X chromosome are used to determine the type of sex chromosomal abnormality.

14. The assay system of claim 2, wherein the frequency of non-pseudoautosomal regions of the Y chromosome are used to determine the type of sex chromosomal abnormality.

15. An assay system for detection of the presence or absence of a fetal sex chromosome aneuploidy in a maternal sample, comprising the steps of:
providing a maternal sample comprising maternal and fetal cell free DNA;
amplifying two or more selected nucleic acid regions from a pseudoautosomal region in the maternal sample;
amplifying two or more selected nucleic acid regions from an autosomal region in the maternal sample;
detecting the amplified nucleic acid regions;
quantifying the relative frequency of the selected nucleic acid regions from the pseudoautosomal and autosomal regions;
comparing the relative frequency of the selected nucleic acid regions from the pseudoautosomal and autosomal regions; and
identifying the presence or absence of a fetal aneuploidy based on the compared relative frequencies of the selected nucleic acid regions.

16. The assay system of claim 15, wherein the maternal sample is maternal blood, maternal plasma or maternal serum.

17. The assay system of claim 15, wherein the maternal sample is maternal plasma.

18. The assay system of claim 15, wherein the relative frequencies of the selected nucleic acid regions are individually calculated, and the relative frequencies of the individual nucleic acid regions are compared to determine the presence or absence of a fetal aneuploidy.

19. The assay system of claim 16, wherein the comparison of the relative frequencies of the pseudoautosomal and autosomal regions is expressed as a chromosomal ratio.

20. The assay system of claim 15, wherein the relative frequencies of the nucleic acid regions are used to determine a chromosome frequency of the pseudoautosomal and autosomal regions, and wherein the presence or absence of a fetal aneuploidy is based on the compared chromosomal frequencies.

21. The assay system of claim 15, wherein the quantified relative frequencies of the selected nucleic acid regions are normalized following detection and prior to quantification.

22. The assay system of claim 15, wherein the selected nucleic acid regions are associated with one or more identifying indices.

23. The assay system of claim 22, wherein the frequencies of the selected nucleic acid regions are quantified based on identification of the one or more associated indices.

24. The assay system of claim 22, wherein the relative frequencies of each nucleic acid region for each chromosome are summed and the sums for each chromosome compared to calculate a chromosomal ratio.

25. The assay system of claim 15, wherein the chromosomal ratio is compared to the mean chromosomal ratio from a normal population and the threshold for identifying the presence or absence of an aneuploidy is at least three times the chromosomal variation in the normal population.

26. The assay system of claim 15, wherein the nucleic acid regions are assayed in a single vessel.

27. The assay system of claim 15, wherein the nucleic acid regions undergo a universal amplification.

28. The assay system of claim 15, wherein the nucleic acid regions are each counted an average of at least 500 times.

29. The assay system of claim 15, wherein the frequency of non-pseudoautosomal regions of the X chromosome are used to determine the type of sex chromosomal abnormality.

30. The assay system of claim 15, wherein the frequency of non-pseudoautosomal regions of the Y chromosome are used to determine the type of sex chromosomal abnormality.

31. An assay system for determination of fetal sex in a maternal sample, comprising:
providing a maternal sample comprising maternal and fetal cell free DNA;
amplifying two or more selected nucleic acid regions from a pseudoautosomal region of a sex chromosome in the maternal sample;
amplifying two or more selected nucleic acid regions from a sex chromosome outside the pseudoautosomal regions;
determining the relative frequency of the selected nucleic acid regions from the sex chromosomes in the maternal sample;
comparing the relative frequency of the selected nucleic acid regions from the pseudoautosomal regions and from the regions outside of the pseudoautosomal regions; and
identifying the fetal sex based on the compared relative frequencies of the selected nucleic acid regions.

32. The assay system of claim 31, wherein the maternal sample is maternal blood, maternal plasma or maternal serum.

33. The assay system of claim 31, wherein the maternal sample is maternal blood.

34. The assay system of claim 31, wherein the relative frequencies of the selected nucleic acid regions are individually calculated, and the relative frequencies of the individual nucleic acid regions of the pseudoautosomal regions and the regions of the sex chromosome outside the pseudoautosomal regions are compared to determine the fetal sex.

35. The assay system of claim 31, wherein the regions from a sex chromosome in the maternal sample outside the pseudoautosomal regions are from the Y chromosome.

36. The assay system of claim 31, wherein the regions from a sex chromosome in the maternal sample outside the pseudoautosomal regions are from the X chromosome.

37. The assay system of claim 31, wherein the selected nucleic acid regions are associated with one or more identifying indices.

38. The assay system of claim 31, wherein the frequencies of the selected nucleic acid regions are quantified based on identification of the one or more associated indices.

39. An assay system for detection of the presence or absence of a sex chromosome aneuploidy comprising the steps of
providing a mixed sample comprising cell free DNA;
sequencing cell-free DNA from the mixed sample;
analyzing the relative frequency of the selected nucleic acid regions from the pseudoautosomal and autosomal regions;
comparing the relative frequency of the selected nucleic acid regions from the pseudoautosomal and autosomal regions; and
identifying the presence or absence of an aneuploidy of a sex chromosome in a cell population based on the compared relative frequencies of the pseudoautosomal and autosomal regions.
